(19) United States
(12) Patent Application Publication
(10) Pub. No.: US 2022/0273363 A1
(43) Pub. Date: Sep. 1, 2022

(54) EXPANDABLE ABLATION DEVICES AND METHODS OF USE

(71) Applicant: Boston Scientific Scimed, Inc., Maple Grove, MN (US)

(72) Inventors: Serena SCOTT, Worcester, MA (US); Mingxiang XU, Wayland, MA (US); Carolina VILLARREAL, Hopedale, MA (US); Hong CAO, Maple Grove, MN (US); Christopher WATSON, Lincoln, MA (US)

(73) Assignee: Boston Scientific Scimed, Inc., Maple Grove, MN (US)

(21) Appl. No.: 17/632,829

(22) PCT Filed: Aug. 10, 2020
(86) PCT No.: PCT/US2020/045602

§ 371 Date: Feb. 4, 2022

Related U.S. Application Data
(60) Provisional application No. 62/885,867, filed on Aug. 13, 2019.

(51) Int. Cl. A61B 18/14

(52) U.S. Cl. CPC A61B 18/1492; A61B 2018/00577

(57) ABSTRACT

A medical system having a medical tool, including a handle, a shaft extending from the handle and defining a lumen, a wire attached to and extending from the handle through the lumen, expandable elements at a distal end of the wire. An electrical generator is coupled to a proximal end of the wire and supplies a first waveform to the wire and the expandable elements as the expandable elements expand from an unexpanded state to an expanded state within a tissue. The electrical generator supplies a second waveform to the wire and the expandable elements when the expandable elements are in the expanded state within the tissue. The first waveform cuts tissue, the second waveform ablates tissue using radiofrequency ablation or irreversible electroporation.

### CROSS-REFERENCE TO RELATED APPLICATIONS

This application claims the benefit of priority from U.S. Provisional Application No. 62/885,867, filed Aug. 13, 2019, which is incorporated by reference herein in its entirety.

### TECHNICAL FIELD

The present disclosure relates generally to endoscopic medical devices and methods of use. More particularly, in some embodiments, the disclosure relates to endoscopic medical tools and methods related to accessing target sites and cutting and/or applying energy to the target sites.

### BACKGROUND

Medical tools for applying energy to target tissue, for example to ablate tissue, may include a needle probe or a wire probe, and the shape of the probe generally does not conform to the size and/or shape of the targeted tissue. Drawbacks of many endoscopic procedures using such tools include, for example, the inability to damage or destroy a target tissue without having to maneuver the distal tip of the tool throughout a procedure. Many conventional tools also generate a generally ellipsoidal ablation zone. If the target site does not match the size of the ellipsoid, or if the target site is a shape other than an ellipsoid, the tool must be moved to different locations of the target site to damage or completely destroy the targeted tissue. Movement of the tool can result in ablating non-targeted tissue and/or causing trauma to tissue surrounding the target site. The present disclosure may solve one or more of these problems or other problems in the art. The scope of the disclosure, however, is defined by the attached claims and not the ability to solve a specific problem.

### SUMMARY OF THE DISCLOSURE

A medical system, comprising, a medical tool having a handle, a shaft extending from the handle and defining a lumen, a wire attached to and extending from the handle through the lumen, and one or more expandable elements at a distal end of the wire. An electrical generator is coupled to a proximal end of the wire, wherein the electrical generator is configured to supply a first waveform to the wire and the one or more expandable elements as the one or more expandable elements expand from an unexpanded state to an expanded state within a tissue site, and the electrical generator is configured to supply a second waveform to the wire and the one or more expandable elements when the one or more expandable elements are in the expanded state within the tissue site, wherein the first waveform is configured to cut tissue and the second waveform is configured to ablate the tissue site using radiofrequency ablation or irreversible electroporation.

Each of the one or more expandable elements may be independently actuatable.

The medical tool may release fluid from a distal end of the shaft at one or more of before, while, and after supplying one or more of the first waveform and the second waveform to the one or more expandable elements.

The shaft may move relative to the wire and the one or more expandable elements, wherein proximal movement of the shaft relative to the wire may expose the one or more expandable elements from a distal end of the shaft.

The system may further include an actuation wire extending in the lumen from the handle to a distalmost end of the medical tool, wherein movement of the actuation wire relative to the shaft exposes the one or more expandable elements from a distal end of the shaft, and wherein movement of the wire relative to the shaft and the actuation wire may expand or retract the one or more expandable elements.

The one or more expandable elements in an expanded state may have a helical shape, a basket shape, or an ellipsoid shape.

The electrical generator may supply the first waveform when the one or more expandable elements collapse from the expanded state to the unexpanded state.

The voltage of the first waveform may be may be greater than or equal to 200 V, and a voltage of the second waveform may be less than or equal to approximately 3.5 kV.

A non-expanding electrode may be provided such that the one or more expandable elements may be arranged about the non-expanding electrode in the unexpanded state.

The cutting waveform may be applied independently to each of the one or more expandable elements.

The one of the one or more expandable elements may be configured to expand a greater distance from a longitudinal axis of the medical tool than another one of the one or more expandable elements.

Each of the one or more expandable elements may include a shape memory metal alloy.

Each of the one or more expandable elements may expand when the first waveform is applied, without any device applying a force to the one or more expandable elements.

A distalmost end of each of the one or more expandable elements may be unattached from a distalmost end of any other expandable element of the one or more expandable elements.

A method of medical treatment, the method comprising advancing a distal end of a medical tool to within a tissue site, retracting an outer sheath of the tool to expose one or more of expandable elements of the medical tool, supplying a first waveform to the one or more expandable elements as the one or more expandable elements expand from an unexpanded state to an expanded state, to cut tissue of the tissue site, supplying a second waveform to the one or more expandable elements when the one or more expandable elements are in the expanded state, wherein the second waveform ablates the tissue.

The first waveform may be independently applied to each of the one or more expandable elements.

Expanding the one or more expandable elements may include independently expanding each of the one or more expandable elements when the first waveform is applied to a corresponding one of the one or more expanding elements to be expanded.

The second waveform may be generated by an alternating current.

The second waveform may be a pulsed direct current waveform.

The method may further include supplying a fluid to the tissue site before, when, or after the second waveform is supplied to each of the one or more expandable elements.

Expanding the one or more expandable elements may include expanding one of the one or more elements a greater distance from a longitudinal axis of the medical tool than another one of the one or more elements.

A voltage of the second waveform may be 20 V to 3.5 kV.

Each of the one or more expandable elements may include a shape memory material, and each of the one or more expandable elements may expand when the first waveform is applied, without any applying a force to the one or more expandable elements.

A method of medical treatment, the method comprising advancing a distal end of a medical tool into tissue, cutting through the tissue by expanding a distal end of the medical tool, wherein a cutting waveform is applied to the distal end during the expanding, and then, ablating the tissue by supplying a radiofrequency ablation waveform or a pulsed irreversible electroporation (IRE) waveform to the distal end of the medical tool.

The distal end of the medical tool may include one or more expandable elements including a shape memory material, and each of the one or more expandable elements may expand when the cutting waveform is applied.

The radiofrequency ablation waveform may be generated by an alternating current, and the IRE waveform may be generated by a direct current.

A voltage of the second waveform may be 20 V to 3.5 kV.

The method may further include supplying a fluid to the tissue at one or more of before, during, or after the ablating step.

The method may further include, upon completion of the ablating step, applying a second cutting waveform to the distal end of the medical tool while retracting the one or more expandable elements.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a perspective view of a medical tool according to an embodiment;

FIGS. 2A-2C are perspective views of an expandable end effector, according to an embodiment;

FIG. 3 is a method for using a medical tool, according to an embodiment;

FIG. 4 is a perspective view of another example of a distal end of a medical tool, according to an embodiment;

FIG. 5 is a perspective view of yet another example of a distal end of a medical tool, according to an embodiment

FIGS. 6A and 6B are perspective views of various expandable end effectors at a distal end of a medical tool, according to embodiments;

FIG. 7 shows perspective views of various expandable end effectors at a distal end of a medical tool, according to an embodiment;

FIGS. 8A-8C are perspective views of an expandable end effector, according to an embodiment;

FIG. 9 is a perspective view of a handle of a medical tool, according to an embodiment; and

FIG. 10 is a method for using a medical tool, according to an example.

### DETAILED DESCRIPTION

The present disclosure is described with reference to an exemplary medical system and medical tool for accessing a target site and applying energy to target tissue to, for example, damage or otherwise destroy the target tissue. However, it should be noted that reference to any particular procedure is provided only for convenience and not intended to limit the disclosure. A person of ordinary skill in the art would recognize that the concepts underlying the disclosed device and application method may be utilized in any suitable procedure, medical or otherwise. The present disclosure may be understood with reference to the following description and the appended drawings, wherein like elements are referred to with the same reference numerals.

For ease of description, portions of the device and/or its components are referred to as proximal and distal portions. It should be noted that the term “proximal” is intended to refer to portions closer to a user of the device, and the term “distal” is used herein to refer to portions further away from the user. Similarly, extends “distally” indicates that a component extends in a distal direction, and extends “proximally” indicates that a component extends in a proximal direction. Further, as used herein, the terms “about,” “approximately” and “substantially” indicate a range of values within +/−10% of a stated or implied value. Additionally, terms that indicate the geometric shape of a component/surface refer only to approximate shapes. For example, an expandable element that is described as having an arc shape or a helical shape indicates that the expandable element has a generally arc or a generally helical shape (e.g., the expandable elements may not form a perfect arc or helix).

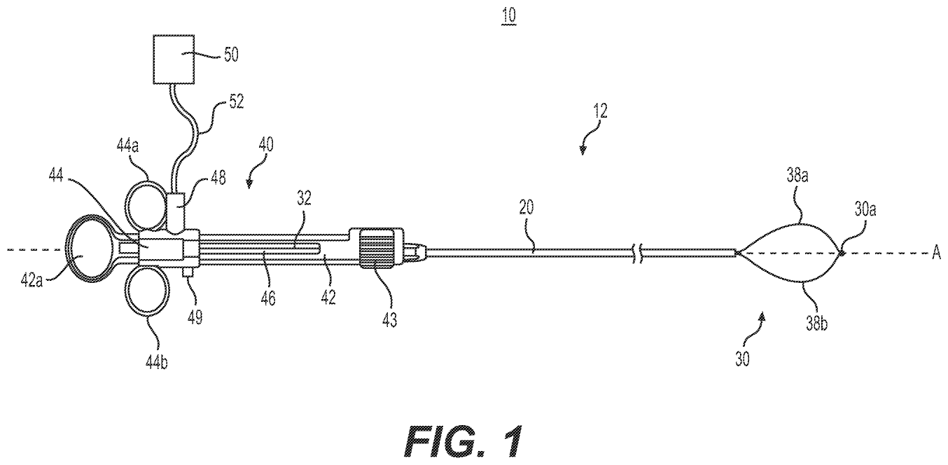

Referring to FIG. 1, a medical system 10 according to an embodiment is shown. Medical system 10 includes a medical tool 12. Medical tool 12 has a shaft (e.g., a flexible sheath or catheter) 20, an end effector (e.g., a probe) 30 at a distal end of shaft 20, and a handle 40. Handle 40 or other similar device is for actuating or controlling end effector 30. Handle 40 is connected to a proximal end of shaft 20.

System 10 also includes an electrical generator 50 provided at a proximal end of medical system 10 near handle 40, to supply electrical current to end effector 30, as will be described herein. For example, electrical generator 50 may be connected to handle 40 via an electrical cord 52 having a distal end coupled to a connector 48 of handle 40, as shown in FIG. 1. Alternatively, electrical generator 50 may be attached directly to a wire 32 to supply electrical current to end effector 30, as will be described herein.

Electrical generator 50 may generate a first waveform W1, e.g., a cutting waveform, suitable for cutting tissue. According to an example, first waveform W1 is a continuous or high duty cycle, e.g., greater than 50%, sinusoidal waveform and may have a frequency of approximately 100 kHz to 5 MHz, and in some embodiments approximately 300 kHz to 500 kHz, and a voltage of first waveform W1 may be approximately 200 V to 2000 V, and in some embodiments approximately 300 V to 700 V.

Electrical generator 50 also may generate a second waveform W2, e.g., an ablation waveform, suitable for ablating tissue. This may be a radiofrequency ablation waveform or a pulsed DC current for irreversible electroporation.

According to an example, second waveform W2 may be a radiofrequency ablation waveform, may be pulsed, and may have a frequency of approximately 100 kHz to 5 MHz, and in some embodiments approximately 300 kHz to 500 kHz, and a voltage of the radiofrequency ablation waveform may be approximately 50 V to 2000 V, and in some embodiments approximate 1000 V to 2000 V with a low duty cycle of less than 50%, and in some embodiments a low duty cycle of approximately 5% to 10%.

Electrical generator 50 may also generate the irreversible electroporation (IRE) waveform, e.g., a direct current, to cause irreversible electroporation to tissue at a target site. Electrical generator 50 may be the same or a different generator than the generator used to generate first waveform W1. IRE is a tissue ablation technique that uses electrical fields to create permanent nanopores in a cell membrane of a tissue, thereby disrupting the cellular homeostasis and damaging or destroying the tissue. IRE may provide a voltage sufficient to generate an electrical field of approximately 600 V/cm to 100,000 V/cm, and in some embodiments an electrical field of approximately 1000 V/cm to 3000 V/cm. The voltage may be applied for more than approximately 50 to 200 pulses, each pulse lasting approximately 40 μs to 4000 μs, and in some embodiments approximately 70 to 100 pulses, each pulse lasting approximately 50 μs to 150 μs. The number of pulses per treatment varies according to the size of the tissue to be treated, for example a tumor.

It will be understood that the same or separate electrical generators may be used to generate each of first waveform W1 for cutting and second waveform W2 for ablation. Further, electrical generator 50 may support cutting, radiofrequency ablation, and/or IRE and may deliver monopolar current and/or bipolar current. As will be described in greater detail herein, each of first waveform W1 and second waveform W2 may be applied to wire 32.

With continued reference to FIG. 1, handle 40 includes a body 42 defining a hole 42a at a proximal end thereof. Shaft 20 is attached at a distal end of body 42, opposite hole 42a. Hole 42a may accommodate a thumb (or a finger) of a user. Handle 40 may be integrally formed with or otherwise fixedly attached to shaft 20. Alternatively, shaft 20 may be rotatable about a rotation mechanism 43, such as a screw mechanism or any other mechanism known in the art. Alternatively, or additionally, shaft 20 may be inserted through the one or more accessory ports in a lumen of a larger endoscope (not shown).

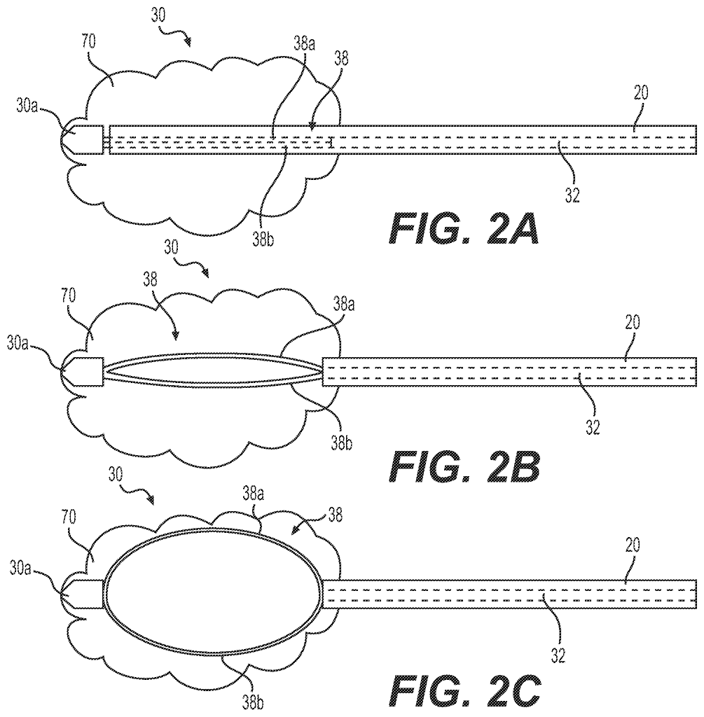

As further illustrated in FIG. 1, a slot 46 extends through body 42 in a direction parallel to a longitudinal axis A of shaft 20 and body 42. A portion of a spool 44 (e.g., a slidable member) is disposed in slot 46 and may move within slot 46 and along body 42 in a direction parallel to longitudinal axis A. As further shown in FIG. 1, spool 44 includes two circular grasping elements 44a, 44b, each having a hole therethrough, extending from spool 44 transverse to longitudinal axis A. Grasping elements 44a, 44b are illustrated as being separated 180 degrees from each other about longitudinal axis A, but the positioning of grasping elements 44a, 44b is not limited thereto. Grasping elements 44a, 44b are grasped by a user to move spool 44 along body 42. For example, a user may place a thumb in hole 42a, an index finger in grasping element 44a, and a middle finger in grasping element 44b, allowing the user to move spool 44 along longitudinal axis A. Handle 40 may further include one or more fluid ports 49 for supplying fluid to shaft 20 and/or end effector 30, and/or for attaching a vacuum device thereto to suction liquids and/or other materials from shaft 20 and/or from a target site, such as target tissue 70 in FIGS. 2A-2C.

With continued reference to FIG. 1, wire 32 (the distal end of which is shown in FIGS. 2A-2C) is connected to and extends distally from the distal end of spool 44. Wire 32 extends through a hole (not shown) at the distal end of body 42 and into a lumen (not shown) of shaft 20. As will be described in greater detail herein, movement of wire 32 along longitudinal axis A, relative to shaft 20, actuates end effector 30. As will be understood, shaft 20 is a generally circular sheath defining the lumen (not shown) extending from handle 40 along longitudinal axis A, and may be any length suitable for performing a medical procedure.

With reference to FIGS. 1 and 2A-2C, end effector 30 is provided at a distalmost end of wire 32. End effector 30 includes expandable elements 38a, 38b, which form a loop 38 when in an expanded configuration (FIG. 2C). Although expandable elements 38a, 38b are illustrated as identical and opposite one another, it is understood that the expandable elements 38a, 38b may be any shape upon expansion and may be different from each other. A probe tip 30a is at a distalmost end of end effector 30. A diameter of an outermost surface of probe tip 30a may be equal to a diameter of an outermost surface of shaft 20. As will be described herein, this configuration may allow wire 32 to remain within the lumen of shaft 20 during deployment of probe tip 30a into target tissue 70. Probe tip 30a may include a traumatic or an atraumatic tip, but is not limited thereto. In an embodiment, probe tip 30a has a sharp distalmost surface, for example including a needle-like point for ease of insertion into tissue.

While shaft 20 is described as including a lumen (not shown), shaft 20 may include multiple lumens to accommodate other actuators, wires, guidewires, and/or lighting or imaging elements. Additionally, or alternatively, shaft 20 may be placed in another, larger catheter, endoscope, echoendoscope, colonoscope, bronchoscope, ureteroscope, sheath, or other like-device (not shown), if use of tools, suction, light-emitting elements, imaging, or the like associated with the larger device are desired. It will be understood that shaft 20 may include any material known in the art, including, but not limited to, medical grade plastic, metal, or other resin suitable for conducting medical procedures. Further, the material used for shaft 20 may differ depending on the medical therapy being employed. For example, if a radiofrequency ablation technique is being used, shaft 20 may require less electrical insulation than if an irreversible electroporation technique is used. It will be understood, however, that shaft 20 may be designed to have a suitable electrical insulation for either or both the radiofrequency ablation technique or the irreversible electroporation technique.

With reference to FIGS. 2A-2C, an expansion of end effector 30 will be described. Referring to FIG. 2A, end effector 30 is in an unexpanded or collapsed configuration, such that expandable elements 38a, 38b are adjacent each other. A user may use a thumb or finger in hole 42a to draw body 42 proximally while maintaining a position of spool 44, thereby drawing shaft 20 proximally. Moving shaft 20 in a proximal direction relative to wire 32 exposes expandable elements 38a, 38b, as shown in FIG. 2B. The user may alternatively maintain a position of body 42 and move spool 44 proximally using a finger (e.g., an index finger and a middle finger) in each of grasping elements 44a, 44b.

According to an example, end effector 30 may include a shape memory alloy, e.g., Nitinol, or any other electrically conducting material used in conducting medical therapies. It will be understood that this material may be used with any of the expandable elements described herein. In some embodiments, when end effector 30 is disposed within target tissue 70, expandable elements 38a, 38b require the application of first waveform W1 to expand. However, the shape memory material of end effector 30 may allow expandable elements 38a, 38b to self-expand from an unexpanded configuration (FIG. 2A) to an expanded configuration (FIG. 2C) when expandable elements 38a, 38b are exposed from the lumen of shaft 20, without any additional mechanical input from a user, or any other structure or device providing expansion force. As described herein, first waveform W1 may be selectively applied to one or both of expandable elements 38a, 38b, thereby allowing one of expandable elements 38a, 38b to expand independently of the other of expandable elements 38a, 38b.

With reference to FIG. 2C, expandable elements 38a, 38b may expand within target tissue 70 to form an expanded loop 38. It is desirable for the end effector 30 to conform to the size and shape of the target site so that ablation energy can be applied to the entire target site at a same time, thereby reducing procedure times and avoiding potential risks associated with moving the probe.

Sensors, such as a thermal sensor or a thermocouple, may be provided on or near end effector 30 (such as on tip 30a or one or both of expandable elements 38a, 38b) to provide power feedback, prevent overheating, and/or prevent or minimize undesired damage of target tissue 70 and/or surrounding tissue. Further, a position sensor may be provided on medical tool 12 (for example markers on a portion of handle 40, such as body 42) to determine a position of expandable elements 38a, 38b within target tissue 70. Determining a position of expandable elements 38a, 38b, e.g., if expandable elements 38a, 38b are sufficiently expanded, may allow ablation to be automated. For example, if it is determined that expandable elements 38a, 38b have been sufficiently expanded, medical tool 10 may automatically switch from first waveform W1 to a second waveform W2. Additional sensors may also aid a user to determine when end effector 30 is provided within target tissue 70. For example, if end effector 30 is determined to be within target tissue 70, cutting waveform W1 may automatically be applied to wire 32. Handle 40 may also include a feedback, such as a light, haptic feedback, or the like, and/or an actuator/switch. This would allow a user to determine when end effector 30 and/or expandable elements are sufficiently situated in target tissue 70, and to actuate the proper waveform to be applied when appropriate. The medical tool 12 may be used under ultrasound guidance from an echoendoscope.

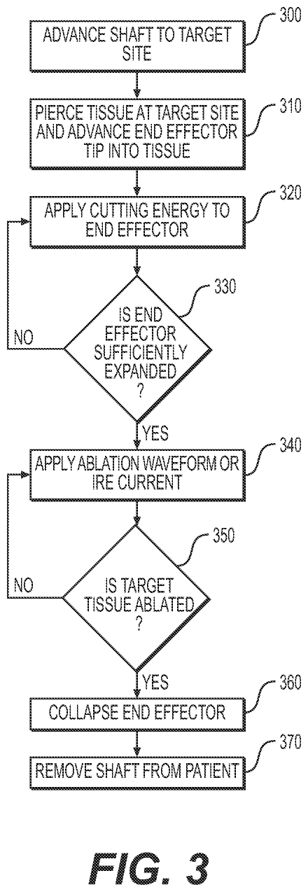

A method of expanding end effector 30 within target tissue 70, e.g., a tumor, will now be described with reference to FIG. 3. In Step 300, shaft 20 is inserted into a patient and advanced to target tissue 70. As described above, shaft 20 may be inserted directly into the patient, or shaft 20 may be advanced along an endoscope, e.g., an echoendoscope, guidewire, or other like device that has been previously advanced to target tissue 70. In Step 310, probe tip 30a pierces target tissue 70 and advances into the target tissue 70. According to an example, shaft 20 remains disposed about end effector 30 until probe tip 30a reaches a distal end of, or other desired location in, target tissue 70.

After probe tip 30a is positioned at the desired location within target tissue 70, a first waveform W1 is applied to wire 32. Subsequently, spool 44 is maintained in a static position while a user moves body 42, via hole 42a, proximally, causing shaft 20 to be moved in a proximal direction, exposing expandable elements 38a, 38b within target tissue 70, thereby allowing expandable elements 38a, 38b to expand and cut through target tissue 70. In Step 330, it is determined if the expandable elements 38a, 38b are sufficiently expanded to form loop 38. This may be done through ultrasound guidance from an echoendoscope, markings on the device, other imaging modalities, or any other suitable method. If the expandable elements 38a, 38b are not sufficiently expanded, Step 320 is continued. It will be understood that shaft 20 may be moved proximally with respect to end effector 30, thereby exposing expandable elements 38a, 38b prior to applying first waveform W1 to wire 32.

Once expandable elements 38a, 38b are sufficiently expanded, second waveform W2 (e.g., an ablation waveform) is applied to expandable elements 38a, 38b for a predetermined time period and/or until the target tissue 70 is sufficiently damaged or destroyed in Step 340. In Step 350, it is determined if target tissue 70 is sufficiently damaged or destroyed. Temperature sensors and/or electrical impedance sensors on medical tool 12, and/or ultrasound imaging, may be used to evaluate the extent of the damage to target tissue 70. If target tissue 70 is not ablated in Step 350, Step 340 is continued. However, if target tissue 70 is destroyed, expandable elements 38a, 38b are collapsed by pushing body 42 (via hole 42a) of handle 40 in a distal direction while maintaining a static position of spool 44 in Step 360. This causes shaft 20 to move in a distal direction and slide over expandable elements 38a, 38b, forcing expandable elements 38a, 38b into a collapsed configuration. Subsequently, shaft 20 may be removed from the patient in Step 370. Alternatively, the user may remove the device without collapsing the expandable elements.

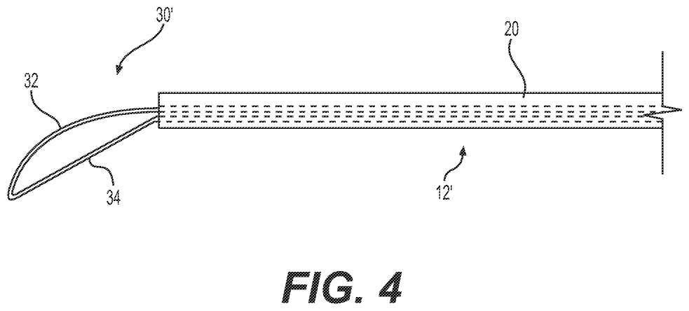

FIG. 4 illustrates another example embodiment of a medical tool 12′. Shaft 20 includes wire 32 extending within a lumen, and actuating wire 34 also extending within the same or different lumen of shaft 20. Actuating wire 34 and wire 32 are attached at distal ends to form an end effector 30′ having a shape, such as a loop, to conform to different target tissue shapes (not shown). Alternatively, wires 32 and 34 are one continuous wire extending from handle 40, such as the handle illustrated in FIG. 1, to the distal end and back to handle 40. As described herein, handle 40 may include actuating elements (not shown), such as a knob or other actuating device, for controlling a movement of wire 32 and actuating wire 34. For example, spool 44 may be split into two independently actuatable members, such that a portion of each member (e.g., one member controlled by grasping element 44a and the other member controlled by grasping element 44b) may slide within slot 46 and parallel to longitudinal axis A. Wire 32 and actuating wire 34 may be advanced along shaft 32 by grasping element 44a and grasping element 44b relative to each other and/or body 42, thereby allowing the distalmost end of wire 32 and actuating wire 34 to penetrate a target tissue. Since actuating wire 34 is attached to the distalmost end of wire 32, movement of actuating by, for example, twisting a knob on handle 40 or moving grasping elements 44a, 44b with respect to each other, a direction of wire 32 and actuating wire 34 may be changed, thereby changing a shape and/or size of end effector 30′. Thus, as a user advances end effector 30′ into a target tissue, the user may direct wire 32 and/or actuating wire 34 to conform to a shape of the target tissue. Once wire 32 is situated within the target tissue, second waveform W2 may be applied to wire 32, which may damage or destroy the target tissue. It will be understood that wire 32 and actuating wire 34 may be formed of a similar or different material. Further, as described herein, deployment of wire 32 into a target site may include applying first waveform W1 to wire 32.

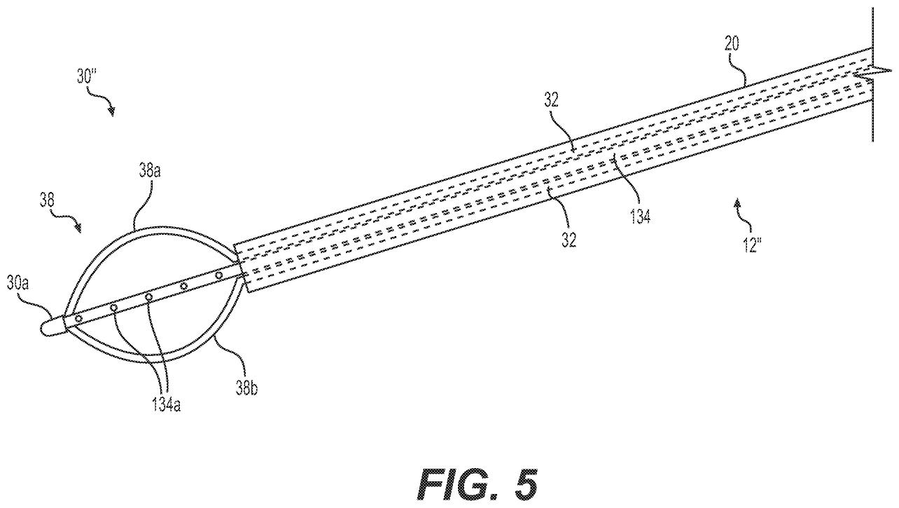

FIG. 5 illustrates another example of a tool 12″ having an end effector 30″. According to an example, tool 12″ includes a tube 134 extending within a central lumen of shaft 20. The central lumen may be the same or a different lumen as the lumen in which one or more wires 32 extend. Tube 134 may convey fluid from a proximal end of medical system 10 (see FIG. 1), e.g., from fluid port 49, to end effector 30″ and tissue site 70. Fluid may be released from tube 134 via one or more holes 134a and/or from a distally-directed opening at a distalmost end of tube 134. According to an example, tube 134 may be attached to probe tip 30a. Alternatively, tube 134 may be unattached from probe tip 30a and may be actuated independently from wire 32. The fluid may include, e.g., saline, drug-containing fluids such as chemotherapeutic agents or immune-oncology agents, or other fluids used in ablation therapies. According to an example, saline may be delivered during thermal ablation to prevent or minimize damage to surrounding tissue and to better conduct electricity, thereby improving the ablation therapy.

End effector 30″ may be deployed in a similar manner as described with respect to FIGS. 2A-2C. When end effector 30″ is deployed within a target tissue, fluid may be released through holes 134a before first waveform W1 is applied, when first waveform W1 is applied to end effector 30″ during expansion of expandable elements 38a, 38b, when second waveform W2 is applied to end effector 30″, and/or after second waveform W2 is applied. While FIG. 5 illustrates a loop 38 and expandable elements 38a, 38b, tube 134 may be used with any end effector described herein. Moreover, while tube 134, and particularly its distal exposed portion, is shown as extending along the longitudinal axis of shaft 20, the position of tube 134 is not limited thereto.

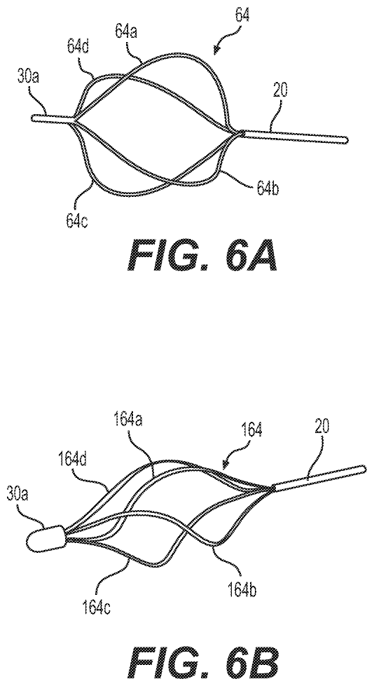

FIGS. 6A and 6B illustrate example end effectors in the shape of baskets 64 and 164. As shown in FIG. 6A, basket 64 includes four expandable elements 64a-64d. Expandable elements 64a-64d may be provided between a distalmost end of wire 32 and probe tip 30a. For example, wire 32 may terminate at, and couple to, a proximal end of expandable elements 64a-64d. Expandable elements 64a-64d may be formed of a shape memory material, e.g., Nitinol, and may self expand without independent actuation when first waveform W1 is applied to the end effector. Alternatively, expandable elements 64a-64d may be individual wires that extend from handle 40 to probe tip 30a. That is, wire 32 may include four separate actuatable wires, one wire corresponding to each of expandable elements 64a-64d. This configuration may allow each of expandable elements 64a-64d to be independently actuatable. In this manner, each expandable element 64a-64d may expand differently to correspond to a size and a shape of target tissue 70. As further shown in FIG. 6A, expandable elements 64a-64d form a generally arc shape in an expanded configuration. According to an example, expandable elements 64a-64d may define an outer circumference of basket 64. As expandable elements 64a-64d are expanded or collapsed, the curvature of expandable elements 64a-64b increases or decreases, respectively. According to another example, expandable elements 64a-64d may form a helix and may overlap in an expanded and/or in a non-expanded state. Additionally, or alternatively, expandable elements 64a-64d may be twisted or bent in an expanded and/or a non-expanded configuration.

FIG. 6B illustrates another embodiment of an end effector in the shape of a basket 164. As with basket 64, basket 164 may be attached to the distal end of wire 32 or may include four independently expandable elements. As shown in FIG. 6B, a shape of each of expandable elements 164a-164d forms a generally helical shape in an expanded configuration. As with basket 64, expandable elements 164a-164d may include a shape memory material and/or may each have a corresponding wire 32 to be individually actuatable.

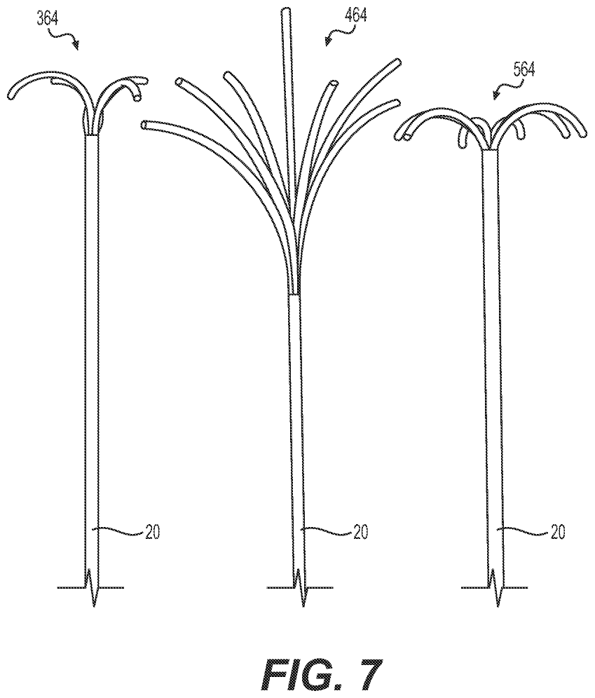

FIG. 7 illustrates three additional examples of end effectors 30. For example, FIG. 7 illustrates expandable members 364, 464, and 564, each of which may extend from a distal end of shaft 20. Expandable members 364 and 564 may have a generally umbrella shape with distalmost ends of one or more expandable elements being proximal of at last a portion of the corresponding elements, when expanded. Expandable member 464 may have a generally tree shape, with a distalmost end of one or more elements being distal of a remainder of the corresponding element, when expanded. The shapes of expandable members 364, 464, and 564 allow target sites 70 having different shapes and sizes to be effectively and efficiently treated. Each of expandable members 364, 464, and 564 may be provided at a distal end of wire 32 and may be deployed as described herein. For example, a user may retract shaft 20 when the end effector is disposed within a target tissue. Expandable members 364, 464, and 564 may be formed of a shape memory material and, when first waveform W1 is applied to the end effector, expandable members 364, 464, and 564 may revert to a predetermined shape, e.g., an umbrella or a tree shape, within target tissue 70. Once deployed, second waveform W2 may be applied to expandable members 364, 464, and 564 as described herein, thereby damaging and/or destroying the target tissue.

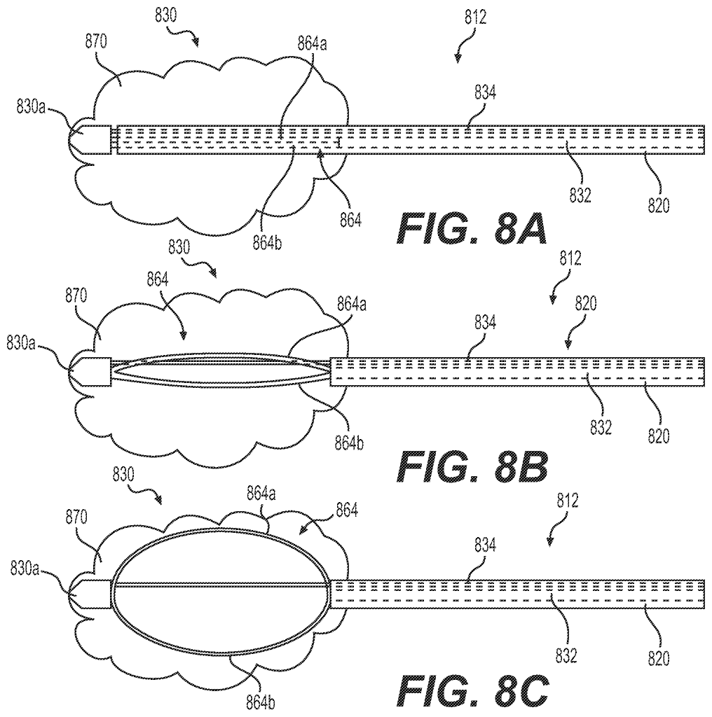

Yet another tool 812 according to an example embodiment is shown in FIGS. 8A-8C. Tool 812 differs from tool 12 in FIGS. 2A-2C in that tool 812 includes two actuating cables or wires 832 and 834. Wire 834 attaches to tip 830a at a distal end of wire 834. End effector 830 of tool 812 includes expandable elements 864a, 864b that form a loop or a basket 864 is an expanded configuration (FIG. 8C). End effector 864 may be attached to a distalmost end of wire 832 or may have separate wires for each of expandable elements 864a, 864b to independently actuate each element of loop or basket 864. End effector 830 includes expandable elements 864a, 864b that may form any shape, e.g., a shape similar to that of end effector 38 in FIG. 2, a basket as shown in FIG. 6A, a helix as shown in FIG. 6B, an umbrella or tree as shown in FIG. 7, or any other suitable shape. A distal end of each element 864a, 864b is attached to tip 830a.

With reference to FIG. 8A, tool 812 may be inserted into target tissue 870 in any manner described herein. Once inserted into target tissue 870, a shaft 820 may be pulled proximally to expose expandable elements 864a, 864b. Shaft 820 may be moved proximally with respect to wires 832 and 834. After exposing expandable elements 864a, 864b, wire 832, which may be connected to expandable elements 864a, 864b, is pushed distally while positions of shaft tip 830a and wire 834, which are connected, remain static. The distal movement of wire 832 causes expandable elements 864a, 864b to expand from a collapsed or unexpanded configuration to an expanded configuration, as shown in FIGS. 8B and 8C. Alternatively, wire 832 and shaft 820 may be connected by a pulley in the handle, such that while shaft 820 retracts, wire 832 is advanced. It will be understood that first waveform W1 is applied to end effector 830 (and particularly wire 832 and elements 864a, 864b) during the expansion of expandable elements 864a, 864b. Once expanded, a second waveform W2 is applied to end effector 830 (and particularly wire 832 and elements 864a, 864b) to damage or destroy target tissue 870. Wire 832 may also be an electrode and may ablate a center of target tissue 870 during activation of second waveform W2.

Once target tissue 870 is sufficiently damaged or destroyed, expandable elements 864a, 864b are collapsed by pulling wire 832 proximally while maintaining a static position of shaft 820 and wire 834. After collapsing expandable elements 864a, 864b, end effector 830 may be retracted into shaft 820. According to an example, shaft 820 may be pushed distally while maintaining a position of both wires 832, 834. Alternatively, a position of shaft 820 and wire 832 may remain static while cable 834 is moved proximally, thereby moving distal tip 830a toward shaft 820 and end effector 830 into shaft 820. Subsequently, shaft 820 may be removed from the patient.

An expanded size of end effector 830 may also be determined and set prior to expanding end effector 830 into target tissue 870. For example, wire 834 may be moved distally, while maintaining a static position of wire 832 and shaft 820, until end effector 830 is sufficiently exposed from shaft 820. After exposing a sufficient amount of end effector 830, e.g., approximately equal to a size slightly smaller than target tissue 870, end effector 830 may be advanced distally into target tissue 870 by moving wire 832, cable 834, and shaft 820 together in a distal direction. Once end effector 830 is disposed in target tissue 870, expandable elements 864a, 864b may be expanded in any manner described herein.

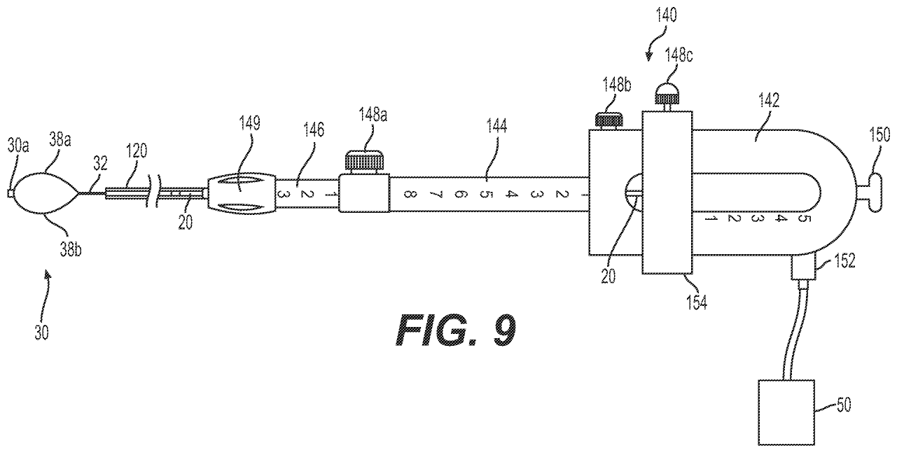

Referring to FIG. 9, a handle 140 according to another example is shown. Handle 140 includes a spool 154 (e.g., a slidable member), a proximal end portion 142, an intermediate portion 144, and a distal end portion 146. Spool 154 is configured to move along handle 140 along an axis extending from proximal end portion 142 to distal end portion 144. Proximal end portion 142 may slide over and around intermediate portion 144, which may in turn slide over and around distal end portion 146. Movement of these portions may provide relative movement of elements at an end effector, as will be described herein. Knobs 148a, 148b, and 148c (e.g., locking mechanisms) may lock corresponding portion of handle 140 to prevent relative movement between spool 154, proximal end portion 142, intermediate portion 144, and/or distal end portion 146, as will be described herein. Knobs 148a, 148b, and 148c may be any locking mechanism, e.g., push buttons, levers, or the like.

According to an example, a sheath 120 is removably attached to a distalmost end of distal end portion 146. Handle 140 may be integrally formed with or otherwise fixedly attached to shaft 20. Handle 140 may contain a locking mechanism 149, such as a screw mechanism or any other mechanism known in the art, thereby allowing handle 140 to be screwed onto an endoscope.

Sheath 120 may surround shaft 20 and may protect an interior surface of the endoscope from wire 32 and/or distal tip 30a during deployment of end effector 30.

Intermediate portion 144 may be advanced or retracted over distal end portion 146 to adjust a distance that sheath 120 extends out a distal end of an endoscope. Knob 148a may lock intermediate portion 144 and distal end portion 146 in a fixed position to prevent relative movement thereof.

Wire 32 may be attached to proximal end portion 142, and may extend through a lumen of shaft 20. An actuation wire may also extend through the same or a different lumen of shaft 20. Shaft 20 is attached to a distalmost end of spool 154. Spool 154 may move shaft 20 independently of the other elements of the device.

Electrical generator 50 may be attached to a connector 152 and a fluid port 150 may allow fluid to be introduced to shaft 20 and/or allow a vacuum to be used to create suction within shaft 20. The electrical generator may be any electrical generator described herein, or may be any electrical generator suitable for supplying the desired waveforms described herein.

As discussed herein, sheath 120, wire 32 and shaft 20 may move independently of each other or, alternatively or additionally, together with each other. For example, maintaining a static position between intermediate portion 144 and distal end portion 146, e.g., by tightening locking knob 148a, may prevent sheath 120 and an endoscope from relative movement. Movement of spool 154 with respect to intermediate end 144 causes shaft 20 to move proximally and distally, thereby exposing expandable elements 64a, 64b and allowing these elements to expand and collapse. Similarly, maintaining a static position between intermediate portion 144 and spool 154 by locking knobs 148b, 148c prevents relative movement between wire 32 and shaft 20.

Sliding spool 154 over proximal end portion 142 and causing spool 154 to move with respect to intermediate portion 144 causes relative movement of shaft 20 with respect to end effector 30, thereby allowing end effector 30 to be exposed or covered. A pulley in handle 140 may connect proximal end of wire 32 to shaft 20, such that retraction of shaft 20 causes wires 64a, 64b to be pushed forward to further expand, while distal tip 30a is fixed in position via a wire 34. For example, movement of proximal end portion 142 while spool 154 is locked to proximal end portion 142 causes tip 30 to be exposed from sheath 120. Knobs 148a, 148b, and 148c may all be tightened to prevent relative movement between distal end portion 146, intermediate portion 144, proximal end portion 142, and spool 154, thereby maintaining a relative position of shaft 20, wire 32, sheath 120, and endoscope (if provided). Indicators, e.g., numbers or other markers, provided on a surface of each of proximal end portion 142, intermediate portion 144, and distal end portion 146 may aid a user to determine a distance proximal end portion 142, intermediate portion 144, distal end portion 146, and spool 154 have moved relative to each other, and thereby aid the user in determining the relative movement of distal tip 30a and shaft 20. It will be understood that indicators are not limited to numbers, and may include any indicator suitable, such as a color, a phrase, etc., for a user to determine relative positions of the various device components.

Handles 40 and 140 may be made of any material known in the art, including, but not limited to, a medical grade plastic or rubber, a ceramic, a metal, or a combination thereof. It will be understood that actuators/handles for use in medical tools of this disclosure are not limited to handle 40 and handle 140, and may be any suitable actuating handle known in the art.

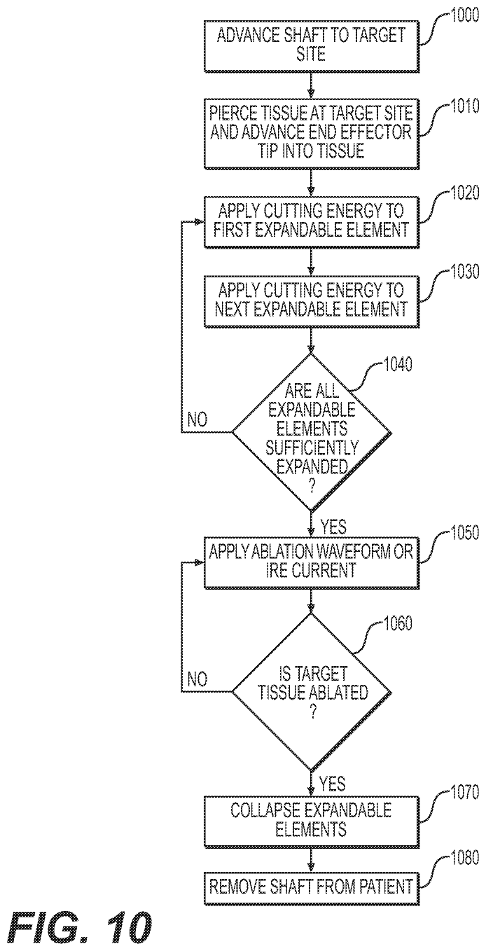

An example method of expanding an end effector at a distal end of shaft 20 and for destroying a target tissue 70, where each expandable element is individually actuated, will now be described with reference to FIG. 10. In Step 1000, shaft 20 is inserted into a patient and advanced to target tissue 70. As described above, shaft 20 may be inserted directly into the patient, or shaft 20 may be advanced within an endoscope, e.g., an echoendoscope, or like device that has been previously advanced to target tissue 70. In Step 1010, probe tip 30a pierces target tissue 70 and advances into target tissue 70. End effector 30 (or any other end effector of this disclosure) may be exposed from shaft 20 and inserted into target tissue 70 by any of the methods described in this disclosure.

After probe tip 30a is inserted into target tissue 70 and end effector 30 is exposed within target tissue 70, expandable elements of end effector 30 are expanded. As an example, description will be made with reference to expandable elements 64a-64d of FIG. 6A, but the method will apply equally to any end effector of this disclosure. In Step 1020, first waveform W1 is transmitted along wire 32 to first expandable element 64a. In Step 1030, first waveform W1 is transmitted along an expandable element adjacent first expandable element 64a in a clockwise direction, e.g., second expandable element 64b. It will be understood that the adjacent expandable member may be in a counterclockwise direction, or a different pattern of selecting the expandable element may be determined based on the size or shape of target tissue 70. Further, in the case where target tissue 70 is abnormally shaped, one or more of expandable elements 64a-64d may be expanded to an extent greater than other expandable elements 64a-64d, thereby expanding to a shape of target tissue 70. In an example, each expandable element may be electrically insulated relative to other expandable elements, to permit energy transfer to an element independent of other elements.

With continued reference to FIG. 10, it is determined in Step 1040 if the expandable elements 64a-64d are sufficiently expanded. If the expandable elements 64a-64d are not sufficiently expanded, Steps 1020 and 1030 are continued. Once expandable elements 64a-64d are fully expanded, second waveform W2 may be applied to expandable elements 64a-64d for a predetermined time period and/or until target tissue 70 is sufficiently damaged or destroyed in Step 1050. Temperature and/or electrical impedance sensors on medical tool 12, and/or ultrasound imaging, may be used to evaluate the extent of damage to target tissue 70. In Step 1060, it is determined if target tissue 70 is sufficiently damaged or destroyed. If so, expandable elements 64a-64d are collapsed, and basket 64 is moved into shaft 20 in Step 1070. Alternatively, the user may remove the device without collapsing the expandable elements. If target tissue 70 is not destroyed, Step 1050 is continued, in any of the manners disclosed herein.

While different end effectors have been described, including different shapes thereof, it will be understood that the shape of these end effectors and expandable members are not limited. Moreover, a size and shape of end effectors may be selected based on a shape of target tissue. As described herein, expandable elements may be expanded different amounts, e.g., different distances from longitudinal axis A, thereby allowing end effector 30 to conform to a shape of target tissue 70. Further, wire 32 and/or any of the expandable elements described herein, e.g., expandable elements 64a-64d, may be formed of Nitinol or any other self-expanding material. Alternatively, wire 32 may be formed of a first material and the expandable elements may be formed of a second material (similarly, any wires and/or expandable elements described herein may be formed of an electrically conductive material that is not self-expanding). To ensure electric conductivity, wire 32 and the expandable elements disclosed herein may be formed of any material suitable to conduct electricity and for use in cutting and ablation therapies.

It will be apparent to those skilled in the art that various modifications and variations can be made to the disclosed device without departing from the scope of the disclosure. For example, the configuration of baskets and expandable elements may be altered to suit any medical tool and/or target site. It will be understood that any handle suitable for use in deploying a wire or a basket in a medical therapy may be used with the shaft, and/or the shaft may be used with any endoscope used in medical therapies. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

1-15. (canceled)

16. A method of medical treatment, the method comprising:
advancing a distal end of a medical tool to within a tissue site;
retracting an outer sheath of the tool to expose one or more of expandable elements of the medical tool;
supplying a first waveform to the one or more expandable elements as the one or more expandable elements expand from an unexpanded state to an expanded state, to cut tissue of the tissue site; and
supplying a second waveform to the one or more expandable elements when the one or more expandable elements are in the expanded state, wherein the second waveform ablates the tissue using radiofrequency ablation or irreversible electroporation.

17. The method according to claim 16, wherein the first waveform is independently applied to each of the one or more expandable elements.

18. The method according to claim 17, wherein expanding the one or more expandable elements includes independently expanding each of the one or more expandable elements when the first waveform is applied to a corresponding one of the one or more expanding elements to be expanded.

19. The method according to claim 16, wherein the second waveform is generated by an alternating current.

20. The method according to claim 16, wherein the second waveform is a pulsed direct current waveform.

21. The method according to claim 16, further comprising:
supplying a fluid to the tissue site at one or more of before, during, and after expansion of the expanding elements.

22. The method according to claim 16, wherein expanding the one or more expandable elements includes expanding one of the one or more elements a greater distance from a longitudinal axis of the medical tool than another one of the one or more elements.

23. The method according to claim 16, wherein a voltage of the second waveform is 20 V to 3.5 kV.

24. The method according to claim 16, wherein each of the one or more expandable elements includes a shape memory material, and each of the one or more expandable elements expands when the first waveform is applied, without any applying a force to the one or more expandable elements.

25. A method of medical treatment, the method comprising:
advancing a distal end of a medical tool into tissue;
cutting through the tissue by expanding a distal end of the medical tool, wherein a cutting waveform is applied to the distal end during the expanding; and
then, ablating the tissue by supplying an ablation waveform to the distal end of the medical tool.

26. The method according to claim 25, wherein the distal end of the medical tool includes one or more expandable elements including a shape memory material, and wherein each of the one or more expandable elements expands when the cutting waveform is applied.

27. The method according to claim 25, wherein the ablation waveform includes a radiofrequency ablation waveform or an irreversible electroporation (IRE) ablation waveform, wherein the radiofrequency ablation waveform is generated by an alternating current, and wherein the IRE ablation waveform is generated by a direct current.

28. The method according to claim 27, wherein a voltage of the ablation waveform is 20 V to 3.5 kV.

29. The method according to claim 28, further comprising:
supplying a fluid to the tissue at one or more of before, during, and after the ablating step.

30. The method according to claim 29, further comprising:
upon completion of the ablating step, applying a second cutting waveform to the distal end of the medical tool while retracting the one or more expandable elements.

31. A medical system, comprising:
a medical tool having:
a handle;
a shaft extending from the handle and defining a lumen;
a wire attached to and extending from the handle through the lumen; and
one or more expandable elements at a distal end of the wire; and
an electrical generator coupled to a proximal end of the wire, wherein the electrical generator is configured to supply a first waveform to the wire and the one or more expandable elements as the one or more expandable elements expand from an unexpanded state to an expanded state within a tissue site, and the electrical generator is configured to supply a second waveform to the wire and the one or more expandable elements when the one or more expandable elements are in the expanded state within the tissue site, wherein the first waveform is configured to cut tissue, and the second waveform is configured to ablate tissue using radiofrequency ablation or irreversible electroporation.

32. The medical system according to claim 31, wherein a voltage of the first waveform is greater than 200 V and a voltage of the second waveform is 20 V to 3.5 kV.

33. The medical system according to claim 31, further comprising:
a sheath surrounding the shaft; and
an endoscope removably coupled to a distalmost end of the handle, wherein the sheath is provided between an inner wall of the endoscope and a distal tip of the one or more expandable elements.

34. The medical system according to claim 31, further comprising a non-expanding electrode, wherein the one or more expandable elements are arranged about the non-expanding electrode in the unexpanded state.

35. The medical system according to claim 31, wherein each of the one or more expandable elements is independently actuatable.
